(19) United States
(12) Patent Application Publication
(10) Pub. No.: US 2004/0166518 A1
(43) Pub. Date: Aug. 26, 2004

(54) Multilabeling mouse FISH assay for detecting structural and numerical chromosomal abnormalities

(75) Inventors: Andrew J. Wyrobek, Walnut Creek, CA (US); Francesca S. Hill, Livermore, CA (US); Francesco Marchetti, San Ramon, CA (US)

Correspondence Address: John H. Lee, Assistant Laboratory Counsel, Lawrence Livermore National Laboratory, L-703, P.O. Box 808, Livermore, CA, 94551, US

(73) Assignee: The Regents of the University of California

(21) Appl. No.: 10/738,947

(22) Filed: Dec. 16, 2003

Related U.S. Application Data
(60) Provisional application No. 60/437,783, filed on Jan. 2, 2003.

(51) Int. Cl. C12Q 1/68

(52) U.S. Cl. 435/6

(57) ABSTRACT

Described herein is a method for detecting structural and numerical chromosome abnormalities in sperm cells. This invention can also be used to detect chromosome abnormalities resulting from exposure to factors such as chemicals, other materials, radiation and environmental conditions. This invention will allow for the wide spread screening of many different types of factors such as chemical, other materials, radiation and environmental conditions to determine whether they have the chromosome abnormality generating effects

### RELATED APPLICATION

This application is related to Provisional Application No. 60/437,783 filed Jan. 2, 2003 entitled “Mulitlabeling Mouse FISH Assay for Detecting Structural and Numberical Chromosomal Abnormalities” and claims priority thereto under 35 USC 120.

The United States Government has rights in this invention pursuant to Contract No. W-7405-ENG-48 between the United States Department of Energy and the University of California for the operation of Lawrence Livermore National Laboratory.

### BACKGROUND

This invention is a new three-color Fluorescent in Situ Hybridization (FISH) assay, designated as CT8, to detect sperm carrying structural and numerical chromosomal abnormalities. The CT8 assay uses DNA probe sets for two different chromosomes in a sperm cell. The CT8 FISH assay can be used for detecting structural and numerical chromosome aberrations in sperm with widespread applications in genetics, physiology and reproductive toxicology.

### SUMMARY OF THE INVENTION

An embodiment of this invention is a method of detecting abnormalities in chromosomes of cells using a new FISH assay using at least one or more nucleic acid probes such that hybridization between the cell chromosomal DNA of interest and the selected probe set can occur, and wherein the detection of the hybridization shows simultaneously the existence of at least two chromosome abnormalities. This assay can be used to detect three different types of chromosome abnormalities, aneuploidy, diploidy and structural aberrations.

Another embodiment of this invention is a method of screening factors such as chemicals, other materials, radiation and environmental conditions by exposing mice to at least one or more of such factors and detecting abnormalities in chromosomes of the cells of such mice using this new FISH assay to detect whether such factors have chromosome abnormality generating effects.

### BREIF DESCRIPTION OF THE DRAWINGS

The patent or patent application file contains at least one drawings executed in color. Copies of this Patent or Patent Application Publication with color drawing(s) will be provided by the office upon request and payment of the necessary fee.

FIG. 1: Shows chromosomes 2 and 8 of a mouse sperm. A probe with a red rodamine dye is shown at the centromere end of chromosome 2(1). A probe with a green fluorescein dye is shown at the telomere end of chromosome 2(2). A probe with a yellow rodamine plus fluorecein dye is shown at the centromere end of chromosome 8(3). The location of chromosomes 2 and 8 in a mouse sperm with a normal CT8 phenotype is shown (4).

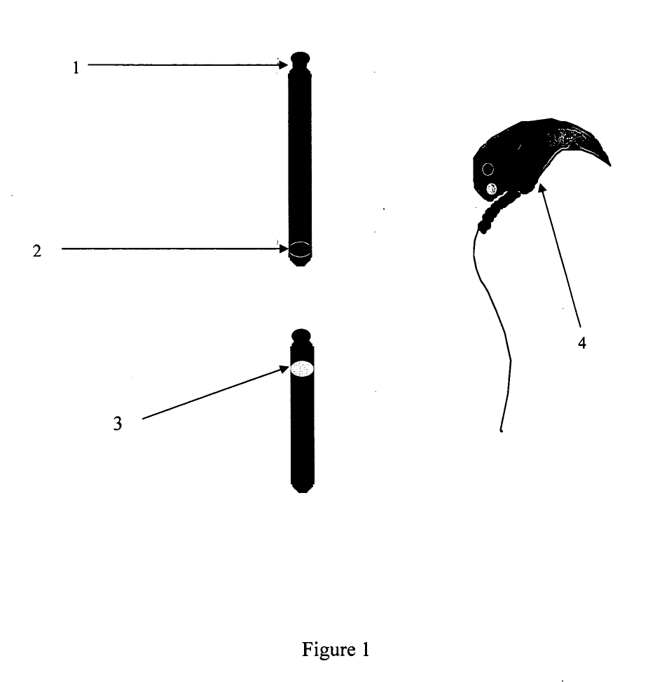

FIG. 2: Photographs of mouse sperm and metaphase II (MII) spermatocytes. a-i. Sperm FISH phenotypes detected with the CT8 assay. The probes for the centromeric region of chromosome 2, telomeric region of chromosome 2, and chromosome 8 are in red, green and yellow, respectively. a. Normal or balanced CT8 sperm. b. Sperm with a duplication of the centromeric region of chromosome 2 (CC T 8). c. Sperm with a deficiency of the centromeric region of chromosome 2 (0 T 8). d. Sperm with a duplication of the telomeric region of chromosome 2 (C TT 8). e. Sperm with a deficiency of the telomeric region of chromosome 2 (C 0 8). ). f. Sperm with disomy 2 (CC TT 8). g. Sperm with disomy 8 (C T 88). h. Sperm with multiple structural abnormalites. There are three signals for the telomeric region of chromosome 2 and only one for the other 2 probes (C TTT 8) i. Diploid sperm (CC TT 88). j-k. MII spermatocyte metaphases showing the products of meiotic segregations from T(2;14) translocation carries after hybridization with chromosome painting probes for chromosome 2 (red) and 14 (green). j. Alternate/adjacent I segregation, 2/2¹⁴ and 14/14². k. Alternate/adjacent I segregation, 2/2¹⁴ and 14²/14/². I. Adjacent II segregation, 2/2¹⁴ and 2/2¹⁴.

### DETAILED DESCRIPTION

De novo aberrations in chromosome structure represent important categories of paternally transmitted genetic damage. Unlike numerical abnormalities, the majority of de novo structural aberrations among human offspring are of paternal origin. Numerical and structural chromosomal abnormalities in germ cells are major contributors to reduced fertility, pregnancy loss and birth defects. Chromosomal abnormalities are present in the majority of miscarriages and in 0.6% of live born infants (Hook, 1983; Shelby et al., 1993). While autosomal aneuploidies (e.g., trisomy 21, 18, and 13) are predominantly maternal in origin, sex chromosomal aneuploidies (e.g., 45,X, 47,XXY, and 47,XYY) have a substantial paternal contribution (Hassold and Hunt, 2001; Hassold et al., 1993; Hecht and Hecht, 1987) and approximately 80% of de novo chromosomal aberrations are paternally derived (Olson and Magenis, 1988).

Multicolor fluorescence in situ hybridization (FISH) has been widely applied to estimate aneuploidy frequencies in human and rodent sperm (Lowe et al., 1998; Lowe et al., 1996; Robbins et al., 1997; Robbins et al., 1993; Rubes et al., 1998) and to detect structural chromosomal aberrations in humans (Sloter et al., 2000; Van Hummelen et al., 1996; Van Hummelen et al., 1997). This invention is a practical assay to detect structural chromosomal aberrations in sperm using FISH. This new FISH assay represents a significant advance for identifying the mechanisms and causes of chromosomal aberrations during spermatogenesis because it allows for the simultaneous identification of at least two different chromosomal abnormalities. This invention is a new three-color sperm FISH assay that uses centromeric and telomeric probes for chromosome 2 and a probe for chromosome 8 for the simultaneous detection of three types of chromosomal damage in mouse sperm: (1) duplications and deletions involving chromosome 2; (2) aneuploidies involving chromosome 2 and 8; and (3) sperm diploidy. This is the first rodent FISH assay for detecting chromosome structural aberrations in sperm and thus is also the first to combine the detection of both numerical and structural chromosomal abnormalities into a simple FISH assay. Thus, this invention greatly increases the speed and efficiency of detecting multiple chromosome aberrations in sperm cells.

Materials and Methods

Animals

Male B6C3F1 mice (Harlan-Sprague-Dawley, Indianapolis, Ind.) 2 to 3 months of age were used for the baseline portion of the study. For validation studies, mice heterozygous for the T(2;14)(E4;E3) reciprocal translocation (Rutledge et al., 1986; Stubbs et al., 1997) were kindly provided by Dr. Lisa Stubbs (LLNL).

Sperm Sample Preparation

Mature sperm from the epididymides of eight B6C3F1 mice, and three mice carrying the T(2;14) reciprocal translocation were isolated and prepared (Lowe, et al., 1996). Briefly, both epididymides were surgically removed, placed into 300 μl of 2.2% sodium citrate at 32° C. and several partial incisions were made with iris scissors (keeping the adjoining tissue intact). After 5 minutes to allow sperm to swim out into the solution, both epididymides were removed from the cell suspension. Seven μl of sperm suspension from each mouse were pipetted onto dry glass slides precleaned with 100% ethanol for at least 24 hours. The cells were smeared over an area of about 22×22 mm using a pipette tip and air-dried overnight. The smears were then used for hybridization or stored at −20° C. in N₂ gas.

Sperm Pretreatment, DNA Probes and FISH

Sperm smears were fixed with 3:1 methanol:acetic acid, air dried and incubated in 10 mM dithiothreitol (Sigma Chemical, St. Louis, Mo.) for 30 minutes on ice followed by a dip in double distilled water at room temperature. The slides were completely air dried and denatured at 78° C. for 8 minutes in 70% formamide (IBI, New Haven, Conn.) and 2×SSC, pH 7.0, and then dehydrated in an ice-cold ethanol series (70%, 85% and 100%), for 2 minutes each. Slides were again air dried prior to hybridization.

Centromeric and telomeric probes for chromosome 2 were identified with support from Applied Genetics Laboratories (Melbourne, Fla.). Yeast artificial chromosomes (YACs) for each probe were purchased from Research Genetics (Huntsville, Md.; mouse chromosome 2 centromere clone ID 460-H-4, Research Genetics Cat. #98022; and mouse chromosome 2 telomere clone ID 121-E-1, Research Genetics Cat. #98022). The clones were cultured according to vendor recommendations and the DNA was isolated via the “Smash and Grab” method (Rose et al., 1990). The probe for chromosome 8 was a combination of two repetitive sequences near the centromere of chromosome 8 (A4-B1), Chrom. 84 and Chrom. 85 (Boyle and Ward, 1992). Plasmid DNA for the chromosome 8 probes were made using a Qiagen Plasmid Kit (Qiagen, Chatswork, Calif.). Probes were labeled with biotin-14-dCTP (2-tel and 8) and/or digoxigenin-11-dUTP (2-cent and 8) by random priming using the BioPrime DNA Labeling System (Invitrogen, Carlsbad, Calif.)

Hybridizations were preformed as previously described (Lowe, et al., 1996) with some modifications. The probe mixture contained 30 μl of mouse Cot-1 DNA (Invitrogen), 2 μl of herring sperm (Invitrogen), and 4μ each of centromere and telomere probes for chromosome 2, and 2 μl of chromosome 8 probes. After ethanol precipitation, the DNA was resuspended in 7 μl of CEP (chromosome enumeration probe) hybridization buffer (Vysis, Downers Grove, Ill.) and 3 μl of distilled water. The probe mixture was denatured at 78° C. for 10 minutes and preannealed for 30 minutes at 37° C. The mixture was applied to the denatured slide, sealed under a coverslip and incubated at 37° C. for ˜48 hrs. The slide was then washed three times in 50% formamide, 2×SSC, pH 7.0, once in 2×SSC and once in PN buffer [0.1 M NaH2PO4/0. 1 M Na2HPO4, pH 8, 0.1% Nonidet P-40 (Sigma Chemical)] for five minutes each at 45° C. A final wash in PN buffer was done at room temperature for 5 minutes. The nuclei were counterstained with 4,6-diamidino-2-phenylindole (DAPI) at 0.1 μg/ml in Vectashield mounting medium (Vector Laboratories, Burlingame, Calif.).

Scoring Criteria and Statistical Analysis

A single scorer analyzed coded slides using a Zeiss Axioplan epifluorescence microscope equipped with single, dual and triple bandpass filters for rhodamine, fluorescein and DAPI as previously described (Lowe, et al., 1996). All data were recorded using the CYTOscore© computer program developed at LLNL. Approximately 10,000 sperm from each of 8 B6C3F1 mice were scored. Each slide was coded a first time and 5,000 sperm were analyzed. After recoding, a second set of 5,000 sperm was analyzed on a different area of the slide. Approximately 1,000 sperm were scored from each of the three T(2;14)(E4;E3) mice. Only hook-shaped nuclei were scored. Sperm were scored as having a normal C-T-8 pattern contained a single red (2 centromere, designated as C in the C-T-8 assay), green (2 telomere, designated as T in the C-T-8 assay) and yellow (8) fluorescent signal. Strict scoring criteria were developed and employed for assigning sperm to four classes of structural aberrations and five classes of numerical abnormalities depending on the number and color of each fluorescent domain contained within each sperm nucleus (Table 1). Sperm nuclei were scored as having two domains of the same loci (e.g., CC-T-8, C-TT-8, C-T-88) if the two fluorescent domains were of similar size, color and separated by a distance larger than that corresponding to the diameter of one domain. Sperm with no detectable fluorescence domains were recorded to assess the efficiency of the FISH procedure.

Cochran's test for equal proportions (Snedecor and Cochran, 1967) was used to compare the results of the 1^{st} and 2^{nd} sets of 5,000 sperm scored for each mouse. If no significant differences were found, the data sets were combined and the frequencies per 10,000 sperm were calculated. Otherwise, the slide was recoded and reintroduced into a pool of slides to be scored. Cochran's test was also used to determine whether the frequencies of duplications and deletions, aneuploidy and diploidy varied significantly among mice of the same genotype.

Meiotic Preparations

Assays using FISH painting on meiotic cells were undertaken to verify the results of the C-T-8 assay results. Meiotic preparations from testes isolated from translocation carriers were made according to Evans et al. (Evans et al., 1964). Slides were air-dried overnight and stored at −20° C. in N₂. Two DNA composite painting probes were used: a biotin-labeled probe specific for the entire chromosome 2 and a FITC-labeled probe specific for the entire chromosome 14 (CAMBIO, Cambridge, UK). Three μl of each DNA probe were suspended in 9 μl of hybridization buffer (CAMBIO) per slide. The hybridization mixture was denatured in, 70% formamide at 75° C. for 5 minutes and subsequently placed at 37° C. for 60 minutes to renature preferentially repeated DNA sequences. Slides were denatured in 70% formamide at 70° C. for 5 minutes and dehydrated in 70% (twice), 90% (twice) and 100% at −4° C. for 3.5 minutes each. The hybridization mixture was applied to each slide warmed to 42° C. for 2 minutes and sealed under a 22×22 mm coverslip with rubber cement. The slides were incubated at 37° C. for ˜48 hr. Post-hybridization washes were performed twice in washing solution (50% formamide, 2×SCC, pH 7) and twice in 0.1×SCC for 5 min each at 45° C. Signals were amplified using the CAMBIO Dual Color Painting Kit (Biotin-Texas Red and FITC) following manufacturer specifications. DAPI diluted to 0.25 μg/ml in Vectashield mounting medium was used as counterstain. At least 50 MII metaphases were analyzed per translocation carrier.

Results

The CT8 FISH assay was developed to detect sperm de novo structural and numerical chromosomal abnormalities in mouse sperm. Sperm showing one copy of each probe, centromeric region of chromosome 2 (C), telomeric region of chromosome 2 (T), and chromosome 8, were scored as normal. The chromosome aberrations detected with this probe combination are: duplications and deletions of the centromeric region of chromosome 2 and duplications and deletions of the telomeric region of chromosome 2. The sperm CT8 assay also detected various numerical abnormalities including sperm: disomy or nullisomy for chromosome 2, disomy or nullisomy for chromosome 8, and diploidy. The latter phenotype may also represent double disomy for chromosomes 2 and 8, but this is expected to be less frequent than diploidy.

Baseline Frequencies in Healthy B6C3F1 Mice

Over 80,000 sperm from 8 adult B6C3F1 mice were analyzed with the CT8 assay to establish the baseline frequencies of sperm carrying various classes of structural and numerical abnormalities (Table 1). The frequency of sperm carrying duplications and deletions of the centromeric region of chromosome 2 were 0.4±0.2 per 10⁴ and 0.6±0.3 per 10⁴, respectively. The frequencies of sperm carrying duplications and deletions of the telomeric region of chromosome 2 were 0.6±0.3 per 10⁴ and 0.9±0.2 per 10⁴, respectively. For both chromosomal regions, the frequencies of sperm carrying duplications and deletions did not differ from a 1:1 ratio. No statistically significant differences were observed among animals.

Average frequencies of sperm disomic for chromosomes 2 or 8 were 0.1±0.1 per 10⁴ and 0.3±0.2 per 10⁴, respectively. Nullisomic frequencies for chromosomes 2 or 8 were 0 per 10⁴ and 1.6±0.5 per 10⁴, respectively. Diploid sperm were the most common type of abnormality detected at a frequency of 5.9±1.1 per 10⁴.

Partial chromosomal duplications and deletions of chromosome 2 occurred at similar frequencies (Table 1) suggesting a common mechanism of formation. These aberrations may be the consequence of unequal crossing over or breakage between the C and T probes before the end of meiosis. Alternatively, duplications and deficiencies can occur via translocations. Stable balanced translocations involving chromosome 2 may occur in stem cells and result in missegregation during meiosis. Because chromosome 2 represents ˜6.4% of the male mouse haploid genome (Disteche et al., 1981), and assuming a random distribution of breaks across the genome, our data suggest that ˜0.4% of mouse sperm carry chromosomal structural aberrations. Although a direct comparison with cytogenetic data in zygotes is not possible because the latter includes types of aberrations that are not detected by the CT8 assay, such as breaks and postmeiotic damage, this estimate is in range with the observed frequencies of zygotes with chromosomal aberrations of paternal origin in untreated mice (Marchetti et al., 2002).

This is the first mouse study to report the sperm disomy frequency for chromosome 2. The frequency of sperm hyperhaploid for chromosome 8 found in this study is below the prior range (2 to 8 per 10⁴) obtained in previous studies from our laboratory using the same probe (Baulch et al., 1996; Lowe et al., 1995; Lowe, et al., 1996; Schmid et al., 2001). However, the method developed for the CT8 assay does not use lithium diiodosalicylate for further decondensation of the sperm DNA. It is possible that over-decondensation in prior studies lead to probe signal splitting between the two clones used for the 8 probe and artificially increased the number of sperm with 2 signals for chromosome 8. Extrapolating the frequencies of disomic sperm for chromosome 2 and 8 collected with the CT8 assay to the haploid genome, the data suggests that 0.1% of mouse sperm are hyperhaploid. This frequency is in line with the 0.3% reported in MII spermatocytes (Miller and Adler, 1992).

Baseline Frequencies in T(2;14) Mice

Three T(2;14) reciprocal translocation carriers were used to validate the ability of the CT8 assay to detect duplications and deletions involving chromosome 2. These mice are predicted to produce high frequencies of sperm carrying such aberrations. Over 43% of the sperm of T(2; 14) carriers showed chromosomal aberrations involving chromosome 2 by the CT8 assay (Table 2). Sperm with the normal sperm FISH phenotype, i.e. CT8, may represent either a normal or a chromosomally balanced sperm generated by alternate segregation. Because we did not use probes for both chromosomes involved in the translocation, the same sperm FISH phenotype may have originated from 2:2 or 3:1 segregations. Only the product of a 4:0 segregation could have been unequivocally identified but none were found.

Duplications and deletions of the centromeric region of chromosome 2 occurred at frequencies of 2.1% and 2.0%, respectively. As expected, duplications and deletions involving the telomeric region were much more common and represented 19% and 20% of the sperm analyzed, respectively. Duplications and deletions of the same chromosomal region of chromosome 2 are reciprocal products of meiotic alternate/adjacent I segregation and are expected to be produced at equal frequencies; indeed their observed frequencies were similar.

Disomy and nullisomy for chromosome 2, and sperm with multiple structural aberrations, which were most likely generated from adjacent II segregation, were 0.6, 0.7%, and 1.1% respectively.

Analysis of Meiotic Preparations in T(2;14) Mice

To further validate the CT8 assay through comparison of the frequency of chromosome anomalies at Meiosis II (MII) with those seen in sperm, over 200 MII spermatocyte metaphases from T(2;14) translocation carriers were analyzed by dual-color FISH with chromosome painting probes for the entire chromosome. The use of chromosome painting allowed the identification at MII of all products of meiotic chromosome segregation (Tease, 1996; Tease, 1998). In addition to the normal (2 and 14) and translocated (2¹⁴ and 14²) chromosomes, recombinant chromosomes (2/2¹⁴ and 14/14²) produced by chiasmata in the interstitial pairing segments were easily identified. Translocation segregation products were classified as alternate (balance segregation of nonhomologous centromeres), adjacent I (unbalanced segregation of nonhomologous centromeres) or adjacent II (segregation of homologous centromeres).

As shown in Table 3, alternate and adjacent I segregants, which could not be discriminated due to the occurrence of crossing over events, represented 88.0% of the metaphases analyzed. Meiotic metaphases produced by adjacent II and 3:1 segregation were 11.1% and 0.9%, respectively.

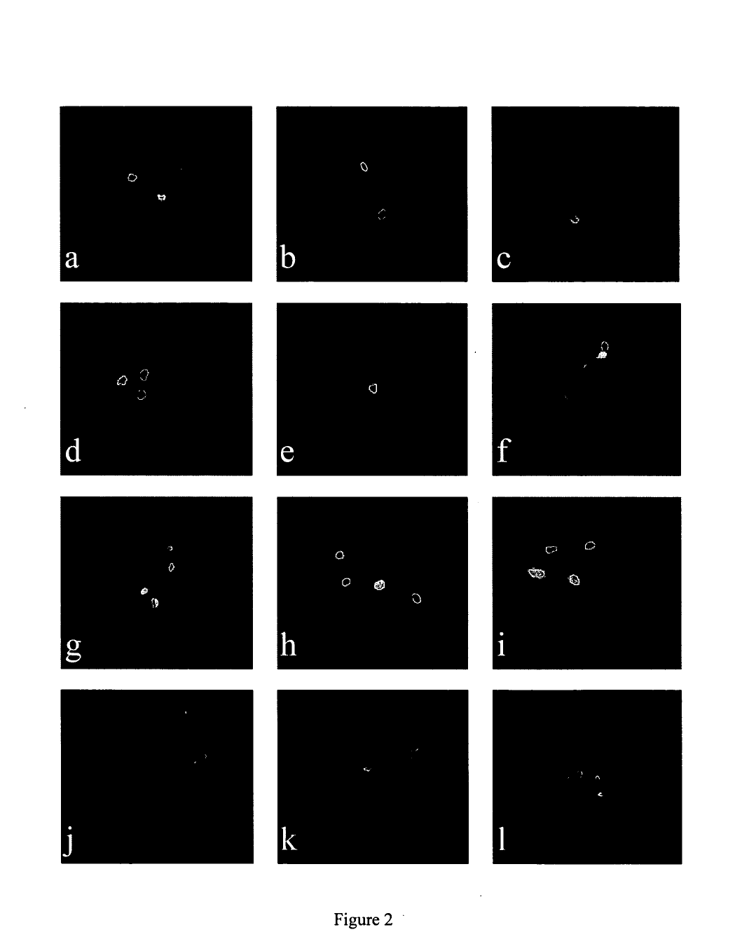

The relative frequencies of alternate, adjacent I and adjacent II segregation types observed in MII metaphases of T(2;14) carriers (Table 3) were used to predict the frequencies of normal and unbalanced sperm that would be detected by the CT8 assay. For example, the MII spermatocyte depicted in FIG. 2(1) would be expected to produce a CCTT8 and a CC08 sperm if the two normal 2 chromatids segregated together, or two CCT8 sperm if each normal 2 chromatid segregated with the recombinant 2¹⁴ chromatid of the homologue. The predicted frequencies of sperm FISH phenotypes produced in these translocation carriers based on the numbers and types of MII spermatocytes (Table 3) were compared with the frequencies observed by the CT8 assay (Table 2). As shown in Table 4, there was good agreement between the MII predictions and the observations made by the CT8 sperm assay for all the FISH phenotypes. These results show that the CT8 assay reliably detects sperm with chromosome structural aberrations.

As expected, the frequencies of sperm with chromosome structural aberrations were significantly increased in the translocation carriers. FISH phenotypes originating from alternate and adjacent I segregation (CTT8 and C08) were more common than those originating from adjacent II segregation (CCT8 and 0T8). This was confirmed by the analysis of MII spermatocytes that showed that adjacent II segregation products represented only 11% of the metaphases analyzed. This is in agreement with predictions based on the location of the breakpoints in human translocations indicating that when the ‘sum of the lengths of centric fragments’ is greater than the ‘sum of the lengths of the translocated segments’ adjacent I segregation is highly favored (Jalbert et al., 1980).

Our study of sperm from healthy mice showed that: 1) the spontaneous frequencies of chromosome structural aberrations were higher than those of numerical abnormalities for the same chromosome; 2) the frequencies of sperm with duplications and deletions are symmetrical for both the telomeric and centromeric regions and occurred both similar frequencies; and 3) diploidy was the most common chromosome abnormality in healthy mice. Two factors validate the accuracy of the CT8 assay for detecting chromosome structural aberrations. First, sperm FISH phenotypes that are the reciprocal products of the same meiotic segregation event were detected at similar frequencies. Secondly, the types and frequencies of abnormal sperm detected in T(2;14) mice by the CT8 FISH assay were in good agreement with the expected frequencies based on the analysis of MII spermatocyte metaphases. Comparisons with data obtained with similar FISH assays for both chromosomal aberrations and aneuploidy in humans suggest that the spontaneous frequencies of sperm with structural aberrations and aneuploidy in mice is more than 5-fold lower than that in humans.

Comparison of Structural and Numerical Abnormalities in Healthy Men and Mice

The development of the CT8 assay now offers the possibility of interspecies comparisons of the baseline frequencies of chromosome structural aberrations in human and mouse sperm (Table 5). The human assays use a probe combination similar to the one used in our mouse assay, namely, a probe for the telomeric region of an autosomal chromosome (specifically, chromosome 1) and another probe for the centromeric region of the same chromosome. A probe for chromosome 8 was used by Van Hummelen et al. (Van Hummelen, et al., 1996), one for chromosome 16 was used by Baumgartner et al. (Baumgartner et al., 1999), while a third probe for chromosome 1 was used by Sloter et al. (Sloter, et al., 2000). In general, the frequencies of sperm carrying duplications and deletions of the telomeric or centromeric regions were higher in human sperm. Extrapolated to the haploid genome, our data suggest that the spontaneous incidence of mouse sperm carrying chromosome structural aberrations are more than 5-fold lower than that found in healthy men. The spontaneous incidence of numerical abnormalities is also substantially lower in mice than in humans. These results are in agreement with the notion that humans have higher incidences of chromosomal imbalances in their germ cells as compared to other mammalian and non-mammalian species (Hassold and Hunt, 2001).

Use of the CT8 Assay to Determine the Chromosome Inducing Effects of Chemicals

In this example of using this assay, the CT8 assay was used to detect the chromosome abnormality generating effects of Etoposide (ET). ET is a chemotherapeutic agent widely used in the treatment of testicular cancer that has been shown to induce chromosomal structural aberrations and aneuploidy in male meiotic germ cells. The CT8 assay was used to study the induction of chromosomal abnormalities in the sperm of mice treated with 80 mg/kg of ET. Semen samples were collected at both 25 and 49 days after ET administration to investigate the effects on pachytene spermatocytes and stem-cell spermatogonia, respectively. CT 8 was also used to study the sperm of mice that had not been exposed to ET. Table 6 shows the results of using the CT8 assay to detect chromosome abnormalities that were induced by the administration of ET and the results of CT8 assays of mice that had not been exposed to ET. ET treatment significantly increased the frequencies of sperm with chromosome structural aberrations after exposure of pachytene sperm-atocytes. Duplications and deficiencies of the centromeric region of chromosome 2 increased 27- and 54-fold, while duplications and deficiencies of the telomeric region of chromosome 2 increased 518- and 188-fold, respectively. Disomy 2 and 8 were increased ˜18-fold with respect to controls. The frequencies of sperm with chromosome structural aberrations and aneuploidy after treatment of stem-cell spermatogonia returned to control levels except for duplications and deletions of the telomeric region of chromosome 2. These results show that exposure to ET has transient effects on chromosomal defects in sperm but may have some long lasting effects on the frequencies of sperm with structural aberrations. This suggests that patients undergoing chemotherapy with ET may remain at higher risk for abnormal reproductive outcomes long after the end of chemotherapy. These results also demonstrate the effectiveness of using the CT8 assay to detect the chromosomal abnormality generating effects of chemicals. Although ET was used in this example, CT8 could be used to detect the chromosome abnormality generating effects of factors other than chemicals such as other materials, radiation and environmental conditions.

These results show that this CT8 mouse sperm FISH assay invention provides an accurate mouse model for characterizing the induction of chromosomal damage (numerical and structural) in mouse. The mouse sperm CT8 assay provides the first robust rodent screen for male germ cell aneugens, clastogens, and agents that may lead to increased risks for chromosomally-based developmental defects and genetic abnormalities in offspring. These results also demonstrate that the CT8 assay can be used to determine the chromosome abnormality generating effects of factors such as chemicals, other materials, radiation and environmental conditions.

References

Baulch J E, Lowe X R, Bishop J B, Wyrobek A J. 1996. Evidence for a parent-of-origin effect on sperm aneuploidy in mice carrying Robertsonian translocations as analyzed by fluorescence in situ hybridization. Mutat Res 372:269-278.

Baumgartner A, Van Hummelen P, Lowe X R, Adler I D, Wyrobek A J. 1999. Numerical and structural chromosomal abnormalities detected in human sperm with a combination of multicolor FISH assays. Environ Mol Mutagen 33:49-58.

Boyle A L, Ward D. 1992. Isolation and initial characterization of a large repeat sequence element specific to mouse chromosome 8. Genomics 12:517-525.

Disteche C M, Carrano A V, Ashworth L K, Burkhart-Schultz K, Latt S A. 1981. Flow sorting of the mouse Cattanach X chromosome, T(X;7)1 Ct in an active or inactive state. Cytogenet Cell Genet 29:189/197.

Evans E P, Breckon G, Ford C E. 1964. An air-drying method for meiotic preparation from mammalian testis. Cytogenetics 3:289-294.

Hassold T, Hunt P. 2001. To err (meiotically) is human: the genesis of human aneuploidy. Nat Rev Genet 2:280-291.

Hassold T, Hunt P A, Sherman S. 1993. Trisomy in humans: incidence, origin and etiology. Curr Opin Genet Develop 3:398-403.

Hecht F, Hecht B K. 1987. Aneuploidy in humans: dimensions, demography, and dangers of abnormal numbers of chromosomes. In Vig B K and Sandberg A A, eds., Aneuploidy. Part II: Incidence and Etiology, New York:Alan R. Liss. pp 9-49.

Hill F S, Marchetti F, Liechty M, Bishop J, Hozier J, Wyrobek A J. 2003. A new FISH assay to simultaneously detect structural and numerical chromosomal abnormalities in mouse sperm.Mol Reprod Dev. 2003 October;66(2):172-80.

Hook E B. 1983. Perspective in mutation epidemiology. 3. Contribution of chromosome abnormalities to human morbidity and mortality and some comments upon surveillance of chromosome mutation rates. Mutat Res 114:389-423.

Jalbert P, Sele B, Jalbert H. 1980. Reciprocal translocations: a way to predict the mode of imbalanced segregation by pachytene-diagram drawing. Hum Genet 55:209-22.

Lowe X, Collins B, Allen J, Titenko-Holland N, Breneman J, van Beek M, Bishop J, Wyrobek A J. 1995. Aneuploidies and micronuclei in the germ cells of male mice of advanced age. Mutat Res 338:59-76.

Lowe X, de Stoppelaar J M, Bishop J, Cassel M, Hoebee B, Moore D I, Wyrobek A J. 1998. Epididymal sperm aneuploidies in three strains of rats detected by multicolor fluorescence in situ hybridization. Environ Mol Mutagen 31:125-132.

Lowe X, O'Hogan S, Moore D 2nd, Bishop J, Wyrobek A. 1996. Aneuploid epididymal sperm detected in chromosomally normal and Robertsonian translocation-bearing mice using a new three-chromosome FISH method. Chromosoma 105:204-210.

Marchetti F, Bishop J B, Cosentino L, Moore II D, Wyrobek A J. 2003. Paternally transmitted chromosomal aberrations in zygotes determine their embryonic fate. Biol. Reprod., published Oct. 29, 2003, 10.1095/Biolreprod. 103.023044.

Miller B M, Adler I D. 1992. Aneuploidy induction in mouse spermatocytes. Mutagenesis 7:69-76.

Olson S B, Magenis R E. 1988. Preferential paternal origin of de novo structural chromosome rearrangements. In Daniel A, ed. The Cytogenetics of Mammalian Autosomal Rearrangements, New York:Liss. pp 583-599.

Robbins W A, Meistrich M L, Moore D, Hagemeister F B, Weier H U, Cassel M J, Wilson G, Eskenazi B, Wyrobek A J. 1997. Chemotherapy induces transient sex chromosomal and autosomal aneuploidy in human sperm. Nature Genet 16:74-78.

Robbins W A, Segraves R, Pinkel D, Wyrobek A J. 1993. Detection of aneuploid human sperm by fluorescence in situ hybridization: evidence for a donor difference in frequency of sperm disomic for chromosomes 1 and Y. Am J Hum Genet 52:799-807.

Rose M D, Winston F, Hieter P. 1990. Methods in yeast genetics: a laboratory course manual. Cold Spring Harbor, N.Y.: Cold Spring Harbor Laboratory Press.

Rubes J, Lowe X, Moore D I, Perreault S, Slott V, Everson D, Selevan S, Wyrobek A J. 1998. Smoking cigarettes is associated with increased sperm disomy in teenage men. Fertil Steril 70:715-723.

Rutledge J C, Cain K T, Cacheiro N L, Cornett C V, Wright C G, Generoso W M. 1986. A balanced translocation in mice with a neurological defect. Science 231:395-7.

Schmid T E, Lowe X, Marchetti F, Bishop J, Haseman J, Wyrobek A J. 2001. Evaluation of inter-scorer and inter-laboratory reliability of the mouse epididymal sperm aneuploidy (m-ESA) assay. Mutagenesis 16:189-95.

Shelby M D, Bishop J B, Mason J M, Tindall K R. 1993. Fertility, reproduction, and genetic disease: studies on the mutagenic effects of environmental agents on mammalian germ cells. Environm Health Perspect 100:283-291.

Sloter E, Lowe X, Moore D I, Nath J, Wyrobek A J. 2000. Multicolor FISH analysis of chromosomal breaks, duplications, deletions, and numerical abnormalities in the sperm of healthy men. Am J Hum Genet 67:862-872.

Snedecor G W, Cochran W G. 1967. Statistical methods, 6th edition. Ames, Iowa: Iowa State University Press.

Stubbs L, Carver E A, Cacheiro N L, Shelby M, Generoso W. 1997. Generation and characterization of heritable reciprocal translocations in mice. Methods 13:397-408.

Tease C. 1996. Analysis using dual-colour fluorescence in situ hybridization of meiotic chromosome segregation in male mice heterozygous for a reciprocal translocation. Chromosome Res 4:61-8.

Tease C. 1998. Chiasma distributions and chromosome segregation in male and female translocation heterozygous mice analysed using FISH. Chromosoma 107:549-558.

Van Hummelen P, Lowe X R, Wyrobek A J. 1996. Simultaneous detection of structural and numerical chromosome abnormalities in sperm of healthy men by multicolor fluorescence in situ hybridization. Human Genet 98:608-615.

Van Hummelen P, Manchester D, Lowe X, Wyrobek A J. 1997. Meiotic segregation, recombination, and gamete aneuploidy assessed in a t(1;10)(p22. 1;q22.3) reciprocal translocation carrier by three- and four probe multicolor FISH in sperm. Am J Hum Genet 61:651-659.

TABLE 1
Frequencies of sperm carrying structural and numerical abnormalities in
B6C3F1 mice as determined by the CT8 assay.

| | Fluorescence | Avg. frequency | |
|---|---|---|---|
| Genotype | Pattern^{a} | Total (no. per mouse) | (per 10⁴ ± SE) |
| No. mice | | 8 | |
| No. cells scored | | 80329 | |
| Normal sperm | C T 8 | 80242 | |
| Sperm with structural aberrations: | | | |
| Centromeric duplication | C C T 8 | 3 (1, 1, 0, 0, 1, 0, 0, 0) | 0.4 ± 0.2 |
| Centromeric deletion | 0 T 8 | 5 (2, 0, 2, 1, 0, 0, 0, 0) | 0.6 ± 0.3 |
| Telomeric duplication | C T T 8 | 5 (1, 0, 1, 2, 0, 0, 0, 1) | 0.6 ± 0.3 |
| Telomeric deletion | C 0 8 | 7 (1, 2, 1, 1, 0, 1, 0, 1) | 0.9 ± 0.2 |
| Total structural | | 20 (5, 3, 4, 4, 1, 1, 0, 2) | 2.5 ± 0.6 |
| Sperm with numerical abnormalities: | | | |
| Disomy 2 | C C T T 8 | 1 (1, 0, 0, 0, 0, 0, 0, 0) | 0.1 ± 0.1 |
| Nullisomy 2 | 0 0 8 | 0 (0, 0 , 0, 0, 0, 0, 0, 0) | 0 |
| Disomy 8 | C T 8 8 | 2 (0, 1, 0, 0, 0, 1, 0, 0) | 0.3 ± 0.2 |
| Nullisomy 8 | C T 0 | 13 (1, 1, 5, 1, 1, 2, 0, 2) | 1.6 ± 0.5 |
| Total numerical | | 16 (2, 2, 5, 1, 1, 3, 0, 2) | 2.0 ± 0.5 |
| Diploid sperm | C C T T 8 8 | 47 (7, 7, 9, 9, 8, 3, 2, 2) | 5.9 ± 1.1 |
| Sperm with complex abnormalities: | C T T T 8 8 | 1 (0, 0, 0, 0, 0, 0, 0, 1) | 0.1 ± 0.1 |

^{a}“0” indicates the absence of an expected domain.

TABLE 2
Frequencies of sperm with structural and numerical abnormalities in T(2; 14)
translocation carriers.

| | Fluorescence | Avg. frequency | |
|---|---|---|---|
| Genotype | Pattern^{a} | Total (no. per mouse) | (percent ± SE) |
| No. mice | | 3 | |
| No. cells scored | | 3018 | |
| Normal sperm | CT8 | 1636 (560, 536, 540) | 54.2 ± 0.2 |
| Sperm with structural aberrations: | | | |
| Centromeric duplication | C C T 8 | 62 (22, 22, 18) | 2.1 ± 0.1 |
| Centromeric deletion | 0 T 8 | 60 (17, 20, 23) | 2.0 ± 0.1 |
| Telomeric duplication | C T T 8 | 572 (194, 185, 193) | 19.0 ± 0.1 |
| Telomeric deletion | C 0 8 | 614 (189, 210, 215) | 20.3 ± 0.3 |
| Total structural | | 1308 (422, 437, 449) | 43.3 ± 0.3 |
| Sperm with numerical abnormalities: | | | |
| Disomy 2 | C C T T 8 | 17 (7, 4, 6) | 0.6 ± 0.1 |
| Nullisomy 2 | 0 0 8 | 21 (4, 9, 8) | 0.7 ± 0.1 |
| Disomy 8 | C T 8 8 | 1 (0, 1, 0) | 0.1 ± 0.1 |
| Nullisomy 8 | C T 0 | 0 (0, 0, 0) | 0 |
| Total numerical | | 39 (11, 14, 14) | 1.3 ± 0.1 |
| Diploid sperm | C C T T 8 8 | 2 (1, 1, 0) | 0.1 ± 0.1 |
| Sperm with complex abnormalities: | C C 0 8 | 18 (5, 9, 4) | 0.6 ± 0.1 |
| | 0 T T 8 | 15 (4, 4, 7) | 0.5 ± 0.1 |

^{a}“0” indicates the absence of an expected domain.

TABLE 3
Meiotic segregants in T(2; 14)
translocation carrier mice analyzed in metaphase
chromosomes of secondary spermatocytes.

MII spermatocytes

| Segregation product | Total | % |
|---|---|---|
| Alternate/adjacent I | | |
| 2/2¹⁴ + 14/14² | 169 | 78.2 |
| 2/2 + 14/14 | 1 | 0.5 |
| 2/2 + 14/14² | 7 | 3.2 |
| 2/2¹⁴ + 14²/14² | 4 | 1.9 |
| 2/2¹⁴ + 14/14² | 4 | 1.9 |
| 2¹⁴/2¹⁴ + 14/14 | 2 | 0.5 |
| 2¹⁴/2¹⁴ + 14²/14² | 1 | 0.9 |
| 2/2¹⁴ + 14/14 | 2 | 0.9 |
| Subtotal | 190 | 88.0 |
| Adjacent II | | |
| 2/2¹⁴ + 2/2¹⁴ | 13 | 6.0 |
| 14/14 + 14²/14² | 5 | 2.3 |
| 14/14² + 14/14² | 6 | 2.8 |
| Subtotal | 24 | 11.1 |
| 3:1 segregation | | |
| 2/2¹⁴ + 14/14² + 14/14² | 1 | 0.5 |
| 2/2¹⁴ | 1 | 0.5 |
| Subtotal | 2 | 0.9 |

TABLE 4
Predicted vs. observed percentages of sperm
with structural and numerical abnormalities
in T(2; 14) translocation carrier mice.

| | Fluorescence | Predicted^{a} | Observed^{b} |
|---|---|---|---|
| Genotype | Pattern^{c} | % ± S.E. | % ± S.E. |
| Normal/Balanced sperm | C T 8 | 44.4 ± 1.8 | 54.2 ± 0.2 |
| Sperm with structural | | | |
| aberrations: | | | |
| Centromeric duplication | C C T 8 | 3.0 ± 0.7 | 2.1 ± 0.1 |
| Centromeric deletion | 0 T 8 | 3.7 ± 3.6 | 2.0 ± 0.1 |
| Telomeric duplication | C T T 8 | 22.2 ± 2.8 | 19.0 ± 0.1 |
| Telomeric deletion | C 0 8 | 22.2 ± 1.2 | 20.3 ± 0.3 |
| Total structural | | 51.1 ± 1.9 | 43.3 ± 0.3 |
| Sperm with numerical | | | |
| abnormalities: | | | |
| Disomy 2 | C C T T 8 | 1.5 ± 0.4 | 0.6 ± 0.1 |
| Nullisomy 2 | 0 0 8 | 0.7 ± 0.2 | 0.7 ± 0.1 |
| Disomy 8 | C T 8 8 | 0 | 0 |
| Nullisomy 8 | C T 0 | 0 | 0 |
| Total numerical | | 2.2 ± 0.4 | 1.3 ± 0.1 |
| Diploid sperm | C C T T 8 8 | 0 | 0.1 ± 0.1 |
| Sperm with complex | C C 0 8 | 1.5 ± 0.4 | 0.6 ± 0.1 |
| abnormalities: | 0 T T 8 | 0.7 ± 0.2 | 0.5 ± 0.1 |

^{a}based on meiotic segregants (Table 3)
^{b}from Table 2
^{c}“0” indicates the absence of an expected domain.

TABLE 5
Comparison of the frequencies of sperm
with structural and numerical
abnormalities in mice and men.

| | Mouse | | | |
|---|---|---|---|---|
| | CT8 | Human assays | | |
| | ACM^{c,d} | AM8^{a} | AM16^{b} | |
| Genotype | 10⁴ ± S.E. | 10⁴ ± S.E. | 10⁴ ± S.E. | 10⁴ ± S.E. |
| Sperm with structural | | | | |
| abnormalities: | | | | |
| Centromeric duplication | 0.4 ± 0.2 | 2.1 ± 0.9 | 6.5 ± 2.6 | 0.9 ± 0.4 |
| Centromeric deletion | 0.6 ± 0.3 | 0.7 ± 0.8 | 1.0 ± 1.2 | 0.8 ± 0.3 |
| Telomeric duplication | 0.6 ± 0.3 | 3.2 ± 1.0 | 3.8 ± 1.0 | 4.5 ± 0.5 |
| Telomeric deletion | 0.9 ± 0.2 | 2.9 ± 2.1 | 1.8 ± 1.5 | 4.1 ± 1.3 |
| Genome-wide | 0.4% | 2.2% | 3.2% | 2.6% |
| extrapolation | | | | |
| Sperm with numerical | | | | |
| abnormalities: | | | | |
| Disomy (multiple probe) | 0.1 ± 0.1 | 1.7 ± 1.3 | 5.0 ± 2.4 | 8.9 ± 0.7 |
| Nullisomy (multiple | 0 | 0.4 ± 0.3 | 1.3 ± 1.5 | 1.2 ± 0.8 |
| probe) | | | | |
| Disomy (single probe) | 0.3 ± 0.2 | 1.9 ± 1.3 | 3.5 ± 1.3 | NA |
| Nullisomy (single probe) | 1.6 ± 0.5 | 4.4 ± 2.8 | 3.5 ± 1.3 | NA |
| Genome-wide | 0.1% | 0.4% | 0.9% | NA |
| extrapolation (disomy) | | | | |

^{a}Van Hummelen et al., 1996
^{b}Baumgartner et al., 1999
^{c}Sloter et al., 2000
^{d}This assay does not distinguish between disomy and diploidy

TABLE 6
Comparison of the frequencies of sperm with structural
and numerical abnormalities in normal mice
chromosomes and in B6C3F1 Mice after exposure
to Etoposide detected by the CT8 Assay

| | 25 days | | 49 days | |
|---|---|---|---|---|
| | Controls | Etoposide | Controls | Etoposide |
| Total sperm | 50280 | 45099 | 50444 | 50296 |
| Sperm with | | | | |
| structural | | | | |
| Aberrations, | | | | |
| Chromosome 2: | | | | |
| centromeric | 0.1 ± 0.1 | 4.1 ± 1.1* | 0.1 ± 0.1 | 0.0 ± 0.0 |
| duplication | | | | |
| centromeric deletion | 0.0 ± 0.0 | 2.7 ± 0.7* | 1.7 ± 0.6 | 1.3 ± 0.4 |
| telomeric | 0.1 ± 0.1 | 57.8 ± 9.4** | 0.1 ± 0.1 | 1.6 ± 0.3* |
| duplication | | | | |
| telomeric deletion | 0.3 ± 0.2 | 63.0 ± 12.7** | 0.2 ± 0.1 | 1.6 ± 0.4* |
| Total Structural | 0.5 ± 0.2 | 121.2 ± 19.1** | 2.1 ± 0.6 | 4.5 ± 0.6* |
| Sperm with | | | | |
| numerical | | | | |
| Abnormalities: | | | | |
| dismoy 2 | 0.3 ± 0.2 | 8.9 ± 2.0** | 0.1 ± 0.1 | 0.1 ± 0.1 |
| nullisomy 2 | 0.1 ± 0.1 | 10.6 ± 1.8** | 0.3 ± 0.2 | 0.3 ± 0.2 |
| disomy 8 | 0.2 ± 0.1 | 5.4 ± 1.4* | 0.2 ± 0.1 | 0.2 ± 0.1 |
| nullisomy 8 | 0.6 ± 0.3 | 21.6 ± 3.5** | 0.2 ± 0.1 | 0.2 ± 0.1 |
| Total Numerical | 1.2 ± 0.4 | 45.5 ± 7.4** | 0.8 ± 0.2 | 0.8 ± 0.2 |
| Diploid sperm | 2.4 ± 0.6 | 92.5 ± 12.8** | 0.8 ± 0.2 | 2.5 ± 0.7 |
| Sperm with multiple | 0.0 ± 0.0 | 41.7 ± 4.5** | 0.3 ± 0.2 | 0.7 ± 0.3 |
| abnormalities | | | | |
| (structural and | | | | |
| numerical) | | | | |

Frequencies shown are per 5,000 sperm plus standard error.
*P < 0.01
**P < 0.001

All publications and patents mentioned in the above specification are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in cytogenetics, molecular biology or related fields are intended to be within the scope of the following claims. For example, although an embodiment of this invention uses mouse sperm cells, this invention could be used to detect chromosomal abnormalities in any cells, including human sperm cells and somatic cells.

1. A method of detecting abnormalities in chromosomes of sperm cells comprising:
staining target sperm cell chromosomal DNA with a nucleic acid probe set wherein the target sperm cell chromosomal DNA is stained by contacting the chromosomal DNA with said nucleic acid probe set which comprises a plurality of nucleotide segments complementary to the chromosomal DNA of interest, under conditions appropriate for hybridization of complementary DNA segments and wherein the chromosomal DNA is present in a sperm cell nucleus during the hybridization; and
detecting hybridization between the sperm cell chromosomal DNA and said nucleic acid probe set, wherein the occurrence of hybridization shows the detection simultaneously of at least two types of chromosome abnormalities.

2. The method of claim 1, wherein said chromosome abnormalities detected simultaneously are aneuploidy and structural aberrations.

3. The method of claim 1 wherein said chromosome abnormalities detected simultaneously are diploidy and structural aberrations.

4. The method of claim 1, wherein the probe set comprises a centromeric and a telomeric probe.

5. The method of claim 4, wherein said centromeric probe comprises dioxigenin labeled and said telomeric probe comprises biotin labeled DNA.

6. A method of detecting abnormalities in chromosomes of mouse sperm cells comprising:
exposing one or more mice to at least one factor selected from chemicals, other materials, radiation and environmental conditions;
harvesting sperm cells from said one or more mice;
staining the chromosomal DNA of said sperm cells with a nucleic acid probe set wherein the chromosomal DNA of said sperm cells is stained by contacting the chromosomal DNA with said nucleic acid probe set which comprises a plurality of nucleotide segments complementary to the chromosomal DNA of interest, under conditions appropriate for hybridization of complementary DNA segments and wherein the chromosomal DNA is present in mice sperm cell nuclei during the hybridization; and
detecting hybridization between the chromosomal DNA in said sperm cells and said nucleic acid probe set, wherein the occurrence of hybridization shows the detection simultaneously of at least two types of chromosome abnormalities.

7. The method of claim 6, wherein said chromosome abnormalities detected simultaneously are aneuploidy and structural aberrations.

8. The method of claim 6, wherein said chromosome abnormalities detected simultaneously are diploidy and structural aberrations.

9. The method of claim 6, wherein said probe set comprises a centromeric and a telomeric probe.

10. The method of claim 9, wherein said centromeric probe comprises dioxigenin labeled and said telomeric probe comprises biotin labeled DNA.

11. A method of detecting abnormalities in chromosomes of cells comprising:
staining target cell chromosomal DNA with a nucleic acid probe set wherein the target cell chromosomal DNA is stained by contacting the chromosomal DNA with said nucleic acid probe set which comprises a plurality of nucleotide segments complementary to the chromosomal DNA of interest, under conditions appropriate for hybridization of complementary DNA segments and wherein the chromosomal DNA is present in a cell nucleus during the hybridization; and
detecting hybridization between the cell chromosomal DNA and said nucleic acid probe set, wherein the occurrence of hybridization shows the detection simultaneously of at least two types of chromosome abnormalities.

12. The method of claim 11, wherein said chromosome abnormalities detected simultaneously are aneuploidy and structural aberrations.

13. The method of claim 11, wherein said chromosome abnormalities detected simultaneously are diploidy and structural aberrations.

14. The method of claim 11, wherein the probe set comprises a centromeric and a telomeric probe.

15. The method of claim 11, wherein said centromeric probe comprises dioxigenin labeled and said telomeric probe comprises biotin labeled DNA.

16. A method of detecting abnormalities in chromosomes 2 and 8 of mouse sperm cells comprising:
staining chromosomes 2 and 8 of target mouse sperm cells with a nucleic acid probe set wherein the DNA of chromosomes 2 and 8 are stained by contacting said DNA with said nucleic acid probe set which comprises a plurality of nucleotide segments complementary to the chromosomal DNA of interest in chromosomes 2 and 8, under conditions appropriate for hybridization of complementary DNA segments and wherein the chromosomal DNA is present in chromosomes 2 and 8 of a mouse sperm cell nucleus during the hybridization; and
detecting hybridization between the mouse sperm cell DNA of chromosomes 2 and 8 and said nucleic acid probe set, wherein the occurrence of hybridization shows the detection simultaneously of at least two types of chromosome abnormalities.

17. The method of claim 16, wherein said chromosome abnormalities detected simultaneously are aneuploidy and structural aberrations.

18. The method of claim 16, wherein said chromosome abnormalities detected simultaneously are diploidy and structural aberrations.

19. The method of claim 16, wherein the probe set comprises a centromeric and a telomeric probe.

20. The method of claim 16, wherein said centromeric probe comprises dioxigenin labeled and said telomeric probe comprises biotin labeled DNA.
